(19) United States
(12) Patent Application Publication
(10) Pub. No.: US 2004/0213847 A1
(43) Pub. Date: Oct. 28, 2004

(54) Delayed release pharmaceutical compositions containing proton pump inhibitors

(76) Inventors: Amol Singh Matharu, Bedminster, NJ (US); Mahendra R. Patel, East Brunswick, NJ (US); Mohsen SadatRezaei, Allentown, PA (US); Chuanbin Wu, Franklin Park, NJ (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/421,229

(22) Filed: Apr. 23, 2003

(51) Int. Cl. A61K 9/24; A61K 9/28; A61K 9/14

(52) U.S. Cl. 424/471

(57) ABSTRACT

Thus, the present invention is directed toward an oral pharmaceutical composition in a solid dosage form comprising:

a) a single core comprising a proton pump inhibitor and a lubricant, wherein said single core has an exterior surface;

b) an enteric coating comprising a polymer and a lubricant, wherein said enteric coating is on the exterior surface of said single core, without a separating layer between said single core and said enteric coating; and

c) optionally, a polymer over-coating on said enteric coating.

### FIELD OF THE INVENTION

The present invention relates to new, stabilized delayed release compositions containing proton-pump inhibitors (PPI) from the benzimidazole class of compounds.

### BACKGROUND OF THE INVENTION

Certain benzimidazoles are anti-ulcerous compounds known for decreasing gastric acid secretion. However, these compounds, also known as proton pump inhibitors (PPI), are susceptible to degradation/transformation in acidic reacting and neutral media. In respect to the stability properties of the benzimidazole compounds mentioned above, it is obvious that those in an oral solid dosage form must be protected from contact with the acidic reacting gastric juice and the active substance must be transferred in intact form to that part of the gastrointestinal tract where pH is less acidic, neutral or alkaline and where rapid absorption of the pharmaceutically active substance, i.e., the benzimidazole derivative, can occur. Certain formulations use a separating layer that requires the application of two separate functional coating operations which increases the length of the manufacturing process and the cost of the product. It would desirable to provide an alternative oral dosage composition containing a PPI, that does not rely upon the use of an intermediate or separating layer to stabilize the PPI contained therein.

### SUMMARY OF THE INVENTION

Applicants have developed an oral pharmaceutical composition in a solid dosage form that avoids the need to use a separating layer to separate the core containing the PPI from the enteric coating layer in a tablet dosage form.

Thus, in one embodiment, the present invention is directed toward an oral pharmaceutical composition in a solid dosage form comprising:

a) a single core comprising a proton pump inhibitor and a lubricant, wherein said single core has an exterior surface;

b) an enteric coating comprising a polymer and a lubricant, wherein said enteric coating is on the exterior surface of said single core, without a separating layer between said single core and said enteric coating; and

c) optionally, a polymer over-coating on said enteric coating.

In another embodiment, the present invention is directed towards an oral pharmaceutical composition in a solid dosage form comprising:

a) a single core comprising a proton pump inhibitor and a lubricant, said core being essentially free of an alkaline reacting agent, wherein said single core has an exterior surface;

b) an enteric coating comprising a polymer and a lubricant, wherein said enteric coating is coated onto the exterior surface of said single core, without a separating or intermediate layer between said single core and said enteric coating; and

c) optionally, a polymer over-coating on said enteric coating.

In another embodiment the present invention is directed toward an oral pharmaceutical composition in a solid dosage form comprising:

a) a single core consisting essentially of, as an active ingredient, a proton pump inhibitor, a disintegrant, a filler and a lubricant, wherein said single core has an exterior surface;

b) an enteric coating comprising a polymer and a lubricant, wherein said enteric coating is coated onto the exterior surface of said single core containing said proton pump inhibitor, without a separating or intermediate layer between said single core and said enteric film coating; and

c) optionally, a polymer over-coating on said film coating.

In another embodiment, the present invention is directed towards a process for preparing an oral pharmaceutical composition in a solid dosage form comprising:

a) forming single core comprising a proton pump inhibitor and a lubricant, wherein said single core has an exterior surface;

b) applying a coating of an enteric coating comprising a polymer and a lubricant onto the exterior surface of said single core containing said proton pump inhibitor, and without forming a separating layer between said single core and said enteric coating; and

c) optionally, applying a polymer overcoating on said film coating.

The single core may contain a proton pump inhibitor (PPI) selected from the group consisting of lansoprazole, pantoprazole, rabeprazole, omeprazole, esomeprazole, leminoprazole or mixtures thereof.

The enteric coating may contain a polymer selected from cellulose acetate phthalate, hydroxypropylmethylcellulose acetate succinate (HPMCAS), hydroxypropylmethylcellulose phthalate (HPMCP), polyvinylacetate phthalate, carboxymethylethylcellulose, acrylic acid polymers and co-polymers and methacrylic acid polymers and co-polymers or combinations thereof.

The present invention has the advantage of providing an oral pharmaceutical composition containing a labile PPI in the form of a tablet that can provide improved stability of the PPI contained therein against degradation and/or discoloration by moisture and/or heating.

Another advantage of the present invention is that it provides an oral pharmaceutical composition containing a labile PPI that allows control of the release rate of said labile PPI within wide margins.

Another advantage of the present invention is that it provides an oral pharmaceutical composition and a process for preparation thereof, containing a labile PPI in the form of a tablet that does not require an separating layer to separate the core unit containing the acid-labile PPI from the enteric coating.

Another advantage of the present invention is that it provides a process for preparing an oral pharmaceutical composition, containing a labile PPI in the form of a tablet that can prevent the in situ formation of a separating layer between the core unit containing the acid-labile PPI from the enteric coating.

### IN THE FIGURES

FIG. 1 depicts a single tablet 10 containing a proton-pump inhibitor. Tablet 10 is made up of core 5 and enteric coating 7. Enteric coating 7 is in direct contact with core 5, as illustrated by contact area or surface 61 between enteric coating 7 and core 5. That is, there is no separating layer between core 5 and enteric coating 7.

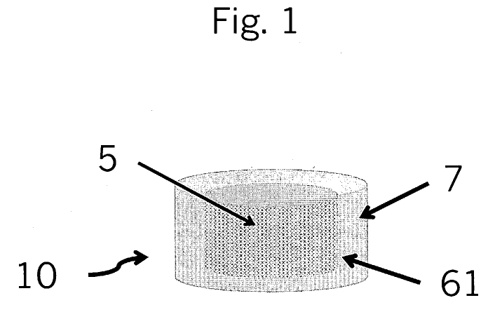

FIG. 2 depicts a capsule 9 containing four tablets or mini-tablets 10a, 10b, 10c and 10d. Capsule 9 is made of two capsule shells 9a and 9b which when assembled together, can hold the tablets or mini-tablets. As discussed in FIG. 1, each tablet or mini-tablet 10a, 10b, 10c and 10d has a core 5 and enteric coating 7.

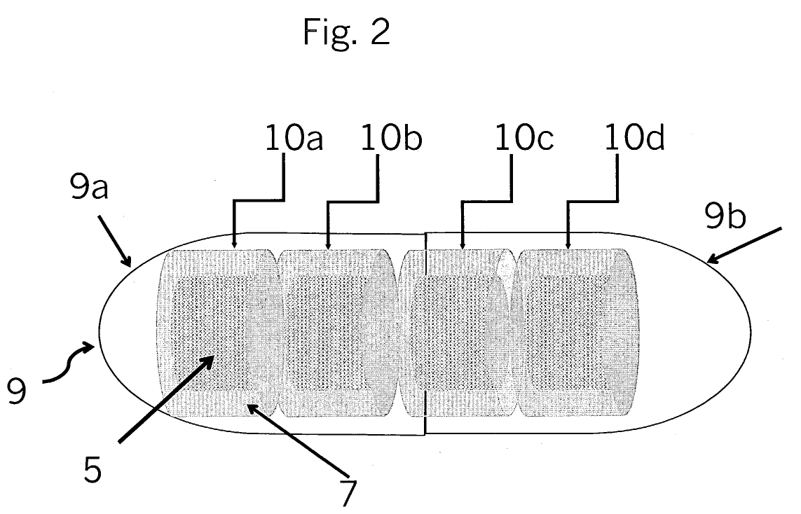

### DETAILED DESCRIPTION OF THE INVENTION

The term “tablet” and “mini-tablet” refer to the solid dosage forms. Generally, a mini-tablet will have less tablet weight and size compared to a tablet.

Utilization of an enteric material as a non-interactive dry application of an otherwise highly interactive material has not been discussed in the prior art, and has not been disclosed in any of the previous patents.

The PPI in an oral solid dosage form should be protected from contact with the acid reacting gastric juice and the active substance should be transferred in intact form to that part of the gastrointestinal tract where the pH is less acidic, neutral or alkaline and where rapid absorption of the pharmaceutically active substance can occur.

A) Core

The terms “tablet core”, “core”, “single core”, “core tablet”, “single tablet core” or “single tablet core unit” have the same meaning and can be used interchangeably. Also, the terms “benzimidazole”, “benzimidazole compound”, “proton pump inhibitor” and “PPI” have the same meaning and can be used interchangeably.

Suitable benzimidazole compounds that can be employed as an active ingredient in the composition of the present invention include those of formula (1)

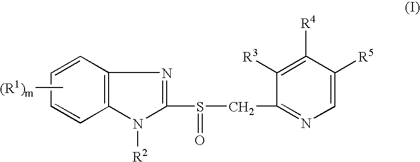

wherein

R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl;

R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl;

R³ and R⁵ are the same or different and each can be hydrogen, alkyl, alkoxy or alkoxyalkoxy;

R⁴ is hydrogen, alkyl, alkoxy which may optionally be fluorinated, or alkoxyalkoxy; and m is an integer of 0 through 4.

Representative examples of such PPIs includes lansoprazole, pantoprazole, rabeprazole, omeprazole, esomeprazole, leminoprazole or mixtures thereof.

The PPIs employed in the present invention may be used in neutral form or in the form of an alkali or alkaline metal salt, such as for instance, the salt of potassium, sodium, lithium, magnesium and/or calcium. Also, the benzimidazole compounds cited above may be used in a neutral form, in a racemic mixture, in the form of a substantially pure enantiomer thereof, as an alkaline salt of the racemic mixture or a single enantiomer, or combinations thereof. The amount of PPI can range from about 5% to about 75% by weight, from about 10% to about 70% by weight or from about 15% to about 60% by weight of the tableted oral pharmaceutical composition. Alternatively, the tablet or capsule can contain a known mass of the PPI, such as 10, 15, 20, 30 or 40 mg.

The term “labile” refers to the property that the PPI are susceptible to degradation in the presence of acid and neutral media, humidity and/or elevated temperatures. For example, degradation of PPI can be catalyzed by acids or acid containing compounds. The PPI may also be unstable in the presence of water or high humidity.

Suitable inert fillers that can be used in the tablet core include lactose, mannitol, starch, sucrose, glucose, hydroxypropyl cellulose, low-substituted hydroxypropyl cellulose, ethylcellulose, hydroxypropyl methylcellulose phthalate, diacetylated monoglycerides, talc, titanium dioxide and other excipients. The amount of filler can range from about 10% to about 90% by weight of the tablet.

Suitable disintegrants that can be used in the tablet core can include sodium starch glycolate or sodium crosscarmellose. The amount of disintegrant can range from about 0.5% to about 30% by weight of the tablet.

Suitable lubricants that can be used in the tablet core or core tablet can include dry or solid lubricants such as magnesium stearate, calcium stearate, silicon dioxide, or sodium stearate and waxes, such as carnauba wax. The lubricant can be employed in both the enteric coating layer and in the core tablet (or tablet core). The percentage of lubricant in either the enteric coating layer or the core tablet can range from about 0.5 to about 30% by weight of each tablet portion, also from about 5 to about 25 percent by weight, also from about 10 to about 15 percent by weight. Such percentage of lubricant can be used in tablets prepared using either a wet granulation method,dry granulation method with absence of water, roller compaction and melt granulation processes.

The PPI is mixed with suitable pharmaceutical constituents, such as those described above for the fillers, disintegrants and lubricants and the resulting mixture is compressed into the tablet core unit. Moreover, the tablet core of the present invention should be essentially free of alkaline reacting agents or compounds, such as those cited in U.S. Pat. No. 6,013,281. The PPI should not be seeded or layered prior to being compressed into the core unit. The size of the formulated core material is approximately between about one and about 20 mm and preferably between about 3 mm and about 15 mm. The manufactured core tablet containing the PPI can be covered with an enteric outer coating or layer. After preparation, the single core tablet has an exterior surface where the enteric outer coating is applied or coated.

B) Enteric Coating or Layer

The terms “enteric coating”, “enteric outer coating”, “enteric layer” or “enteric outer layer” have the same meaning and can be used interchangeably. The enteric coating should be inert or substantially non-interacting with the single, tablet core containing the PPI. The enteric coating may contain ingredients, such as polymers, release rate controlling agents, lubricants, anti-tacking agents, colorants, pigments or other additives to obtain a tablet of good appearance. The amount of enteric coating in the tablet can range from about 0.1 parts to about 3 parts by weight of enteric coating per one part by weight tablet core (about 0.1-3 parts by weight enteric coating:one part tablet core). However, the enteric outer coating does not contain any PPI drug ingredient.

Suitable polymers that can be used in the enteric coating can include film-forming polymers such as anionic co-polymers based on methacrylic acid esters, commercially available as Eudragit L 100 and Eurdragit S 100, trademarks of Rohm, GmbH & Co., K G, Darmstadt, Germany. This enteric coating is insoluble below pH 5 and is thus resistant to gastric fluid. By salt formation in the neutral or weakly alkaline medium of the intestinal fluid, the enteric coating dissolve stepwise at pH values greater than 5.5-7.5. Another suitable polymer that can be used includes HPMCP or HPMCAS, commercially available from the Shin-Etsu Chemical Co. Ltd. A sole polymer can be employed such as HPMCAS or a mixture of polymers can be used, such as Eudragit and HPMCP. Thus, polymers can be cellulose acetate phthalate, HPMCAS, HPMCP, polyvinylacetate phthalate, carboxymethylethylcellulose, acrylic acid polymers and co-polymers and methacrylic acid polymers and co-polymers. The non-interacting property of such enteric coatings can be obtained or enhanced by of the tablet core. The amount of each polymer employed in the enteric neutralizing free acids in the enteric polymer with an inorganic or organic alkaline material, such as sodium hydroxide, magnesium hydroxide, meglumine and the like. The neutralized polymer results in enhanced stabilization coating can range from about 5% to about 99% by weight of the composition.

Suitable release rate controlling agents that can be used in the enteric coating can include lactose, mannitol, starch, sucrose, glucose, hydroxypropylcellulose, low-substituted hydroxypropylcellulose, ethylcellulose, HPMCP, diacetylated monoglycerides, talc or titanium dioxide. The amounts of release agent employed in the enteric coating can range from about 0.5% to about 95% by weight of the composition.

Suitable lubricants that can be used in the enteric coating can include dry or solid lubricants such as magnesium stearate, calcium stearate, silicon dioxide, or sodium stearate and waxes, such as carnauba wax. The lubricant can be employed in both the enteric coating layer and in the core tablet (or tablet core). The percentage of lubricant in either the enteric coating layer or the core tablet can range from about 0.5 to about 30% by weight of each tablet portion, also from about 5 to about 25 percent by weight, also from about 10 to about 15 percent by weight. Such percentage of lubricant can be used in tablets prepared using either a wet granulation method and/or dry blending/compression.

A defoaming agent such as simethicone can also be incorporated into the coating dispersion up to about 5 percent by weight of the enteric coating layer.

A surfactant such as sodium lauryl sulfate can be used as a wetting agent to help disperse the lubricant into the enteric coating suspension or dispersion. The surfactant can be up to about 5 percent of the enteric coating layer.

After the enteric coating is applied to the tablet core, there is no separating layer between said tablet core and the enteric coating.

C. Optional Polymer Over-Coating

Tablets with the enteric coating are then covered with optionally one or more finishing polymer over-coating or tablet film coat(s) or layer(s) to obtain tablets of good appearance, smoothness, color or functionality, such as modified release. The maximum thickness of the applied over-coating layer(s) is normally limited by processing conditions and the desired dissolution or release profile. For example, the tablet film(s) can be a thin coat as compared to the enteric coating. The polymer over-coating can be water soluble or water soluble/swellable in water or have a solubility that is pH dependent. Further, the over-coating can be rapidly disintegrating or even insoluble in water. The materials for the over-coating layer can be pharmaceutically acceptable excipients, such as the same polymers used in the enteric coating layer, sugar, polyethylene glycol, polyvinylpyrrolidone, polyvinyl alcohol, polyvinyl acetate, hydroxypropylcellulose, methylcellulose, ethylcellulose, hydroxypropylmethylcellulose, acrylic acid co-polymers, carboxymethylcellulose sodium, phthalate, HPMCAS, Eudragit (Rohm Pharma Co., West Germany, acrylate co-polymer, amionic in character), polyvinylacetaldiethylaminoacetate, water soluble salts of enteric coating polymers, and waxes, used alone or in mixtures. Additives, such as plasticizers, colorants, pigments, fillers, anti-tacking and anti-static agents, such as for instance magnesium stearate, titanium dioxide, talc and other additives may also be included into the over-coating layer(s). However, the polymer over-coating does not contain any PPI or other active drug ingredient. The amount of polymer coating in the tablet can range from about 0.01 parts to about 1 part by weight of polymer coating per one part by weight tablet core (about 0.01—about 1 part by weight enteric coating:one part tablet core).

The polymer over-coating or tablet film coat can be applied to the enteric coating layered tablet by spraying, coating or layering procedures in suitable equipment, such as coating pan, coating granulator or in a fluidized bed apparatus. In such procedures, water or other solvents may be used to solubilize the materials used for the polymer over-coating or tablet film coat.

The invention can illustrated by the following examples, which are non-limiting as to the scope of the claimed invention.

### EXAMPLE 1

### Lansoprazole Delayed Release (DR) Mini-Tablet

| Mini-tablet core | mg/unit | % w/w |
|---|---|---|
| Lansoprazole | 7.5 | 15 |
| Lactose F.F. | 32.0 | 64 |
| Sodium starch glycolate | 4.5 | 9 |
| Magnesium stearate | 6.0 | 12 |

The mini-tablet core is prepared by dry mixing lansoprazole with lactose F.F., sodium starch glycolate and magnesium stearate. The dry mixture is compressed with a suitable tablet press into 50 mg core tablets containing 7.5 mg of lansoprazole which are 0.1875″ (4.8 mm) in diameter and 0.11″ (2.8 mm) in thickness. In this example, the weight of the mini-tablet is chosen as 50 mg. However, the weight of the mini-tablet could be as low as 5 mg.

| Enteric Coating | mg/unit | % w/w |
|---|---|---|
| Eudragit L30D-55 | 12.6 | 47.0 |
| Low Micron Talc | 6.3 | 23.5 |
| Triethyl Citrate (TEC) | 1.9 | 7.1 |
| Magnesium Stearate | 6.0 | 22.4 |
| Water | 166 | N/A |

The enteric coating is prepared by mixing Eudragit L30D-55, Low Micron Talc, TEC, magnesium stearate and water to form an aqueous mixture or dispersion of the enteric coating. The aqueous mixture or dispersion is sprayed onto the mini-tablet core to form a mini-tablet having an enteric polymer coating. Photographs taken of the cross section of tablet using a scanning electron microscope (SEM) confirmed the absence of an in-situ intermediate layer between the mini-tablet core and the enteric coating.

The release of the drug from the min-tablets is monitored using a dissolution tester, in which 900 mL of simulated gastric fluid (SGF), without enzyme is maintained at 37° C. and used as the dissolution medium for the first 1 hour. The USP 2 dissolution method is used at a rotation speed of 75 rpm. For the next hour, phosphate buffer is used as a media.

Delayed release of lansoprazole is obtained after a period of about 1 hour in SGF dissolution media. The dissolution in phosphate buffer (average, n=6) is as follows:

| Time | % dissolved |
|---|---|
| Media: SGF | |
| 1 hour | 2.4 |
| Media: phosphate buffer | |
| 5 minutes | 25.9 |
| 10 minutes | 60.5 |
| 15 minutes | 68.5 |
| 30 minutes | 75.2 |
| 45 minutes | 77.2 |
| 60 minutes | 79.5 |

### EXAMPLE 2

### Capsule of Lansoprazole Delayed Release (DR) Mini-Tablets

The mini-tablets from Example 1 are filled into size 3 cs white opaque capsules. Each capsule contains two or four mini-tablets.

### EXAMPLE 3

### Pantoprazole Delayed Release (DR) Tablet

The pantoprazole DR tablet has a tablet core, an enteric film coating around the tablet core and a color film coating. The preparation of a 20 mg tablet is described below.

| tablet core | mg/unit |
|---|---|
| pantoprazole sodium | 22.55 |
| magnesium stearate | 4.75 |
| lactose monohydrate | 86.45 |
| crospovidone | 11.25 |

The tablet core is prepared as follows. Dry blend pentaprazole sodium with magnesium stearate to form a first mixture. Dry blend the first mixture with lactose monohydrate and crospovidone to form a second mixture. Dry blend the second mixture with the remaining magnesium stearate to form a tablet core mixture. The lactose monohydrate is a fast flow form. Compress the tablet core mixture using a tablet press into a tablet core. The pantoprazole sodium is in the form of sodium sequihydrate.

The tablet core lacks an alkalizing agent.

| enteric film coating | mg/unit |
|---|---|
| talc | 8.5 |
| magnesium stearate | 4.8 |
| polysorbate 80 | 0.1 |
| methacrylic acid copolymer | 33.3 |
| triethyl citrate | 1.5 |
| simethicone | 0.2 |
| purified water | q.s. |

Coat the tablet core with the enteric film coating as follows. In a first vessel, disperse the talc, magnesium stearate and polysorbate 80 in water using a high shear homogenizer. In a second vessel, stir the methacrylic acid copolymer and add the triethyl citrate to the vortex made by the stirrer. Add the contents of the second vessel to the first vessel while mixing vigorously to form a suspension. Add the simethicone to the suspension and mix. While continuing to stir, spray the suspension onto the tablet core to add the enteric film coating, thus forming a non-colored, pantoprazole tablet. The talc is a low micron or micronized talc. The methacrylic acid copolymer is also known as Eudragit L30-D, type C. Polysorbate 80 is also known as Tween 80 or polyoxyethylenesorbitan monooleat The purified water is used in manufacturing process only and does not appear in final product.

| color film coating | mg/unit |
|---|---|
| opadry brown | 4.500 |
| purified water | q.s. |
| carnauba wax | trace |

Opadry brown is a suspension of hydroxypropylmethylcellulose (HPMC), hydroxypropylcellulose, polyethylene glycol, hydroxypropylcellulose (HPC), polyethylene glycol, titanium dioxide (TiO₂), yellow iron oxide and red iron oxide The non-colored pantoprazole tablet is treated with a commercially available suspension of opadry brown that forms a color film coating on the tablet surface and gives the colored tablet a polished appearance. The colored pantoprazole tablet is imprinted with a trace amount of black ink. The purified water is used in manufacturing process only and does not appear in final product.

### EXAMPLE 4

### Lansoprazole Mini-Tablet for Delayed Release

| Tablet core | Mg/unit | % w/w |
|---|---|---|
| Lansoprazole | 7.5 | 15.0 |
| Lactose F.F. | 30.0 | 60.0 |
| Hydroxypropylmethylcellulose | 2.0 | 4.0 |
| Sodium starch glycolate | 4.5 | 9.0 |
| Magnesium stearate | 6.0 | 12.0 |

The tablet core is prepared by compressing lansoprazole-containing granules. The granules are made of lactose F.F., sodium starch glycolate, hydroxypropylmethylcellulose and magnesium stearate. The granules are made by using wet granulation method and are then dried using either tray drying or fluidized bed drying. The dried granules are compressed with a suitable tablet press into 50 mg core tablets containing about 7.5 mg of lansoprazole which are 0.1875″ (4.8 mm) in diameter and 0.11″ (2.8 mm) in thickness.

| Coating Formulation | Mg/unit | % w/w |
|---|---|---|
| Eudragit L30D-55 | 6.00 | 58.25 |
| Low Micron Talc | 1.60 | 15.53 |
| Triethyl Citrate | 0.90 | 8.74 |
| Simethicone | 0.18 | 1.75 |
| Magnesium Stearate | 1.50 | 14.56 |
| Sodium lauryl sulfate | 0.12 | 1.17 |

The aqueous enteric coating is prepared by spraying the coating dispersion onto core tablets or tablet cores.

The enteric polymer coated tablets are filled into size 3 cs white opaque capsules. Each capsule contains 2 or 4 coated tablets.

The release of the drug from the tablets is monitored using a dissolution tester, in which 900 mL of simulated gastric fluid (SGF), without enzyme is maintained at 37° C. and used as the dissolution medium for the first 1 hour. The USP 2 dissolution method is used at a rotation speed of 75 rpm. For the next hour, phosphate buffer is used as a media.

Delayed release of lansoprazole is obtained after a period of about 1 hour in SGF dissolution media. The delayed release profile of the drug from the capsule is demonstrated by its dissolution in SGF and in phosphate buffer (average, n=6) as follows:

| Time | % dissolved |
|---|---|
| Media: SGF | |
| 1 hour | 0.5 |
| Media: phosphate buffer | |
| 10 minutes | 33.8 |
| 15 minutes | 87.1 |
| 30 minutes | 98.8 |
| 45 minutes | 98.9 |
| 60 minutes | 90.0 |

1. An oral pharmaceutical composition in a solid dosage form comprising:
a) a single core comprising a proton pump inhibitor and a lubricant, wherein said single core has an exterior surface;
b) an enteric coating comprising a polymer and a lubricant, wherein said enteric coating is on the exterior surface of said single core, without a separating layer between said single core and said enteric coating; and
c) optionally, a polymer over-coating on said enteric coating.

2. The composition of claim 1 wherein said enteric coating is film coated onto the exterior surface of said single core.

3. The oral pharmaceutical composition of claim 1 wherein said single core comprises a proton pump inhibitor selected from the group consisting of lansoprazole, pantoprazole, rabeprazole, omeprazole, esomeprazole, leminoprazole or mixtures.

4. The oral pharmaceutical composition of claim 1 wherein said single core comprises a proton pump inhibitor that is lansoprazole or pantoprazole.

5. The oral composition of claim 1 in the form of tablet containing of lansoprazole.

6. The oral composition of claim 1 in the form of tablet containing pantoprazole.

7. The oral pharmaceutical composition of claim 1 wherein said enteric coating comprises a polymer selected from cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers and methacrylic acid polymers and copolymers or combinations thereof.

8. An oral pharmaceutical composition in a solid dosage form comprising:
a) a single core comprising a proton pump inhibitor and a lubricant, said core being essentially free of an alkaline reacting agent, wherein said single core has an exterior surface;
b) an enteric coating comprising a polymer and a lubricant, wherein said enteric coating is coated onto the exterior surface of said single core, without a separating or intermediate layer between said single core and said enteric coating; and
c) optionally, a polymer over-coating on said enteric coating.

9. The composition of claim 8 wherein said enteric coating is film coated onto the exterior surface of said single core.

10. The oral pharmaceutical composition of claim 8 wherein said single core comprises a proton pump inhibitor selected from the group consisting of lansoprazole, pantoprazole, rabeprazole, omeprazole, esomeprazole, leminoprazole or mixtures.

11. The oral composition of claim 8 in the form of tablet containing lansoprazole.

12. The oral composition of claim 8 in the form of tablet containing pantoprazole.

13. The oral pharmaceutical composition of claim 8 wherein said enteric coating comprises a polymer selected from cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers and methacrylic acid polymers and copolymers or combinations thereof.

14. An oral pharmaceutical composition in a solid dosage form comprising:
a) a single core consisting essentially of, as an active ingredient, a proton pump inhibitor, a disintegrant, a filler and a lubricant, wherein said single core has an exterior surface;
b) an enteric coating comprising a polymer and a lubricant, wherein said enteric coating is coated onto the exterior surface of said single core containing said proton pump inhibitor, without a separating or intermediate layer between said single core and said enteric film coating; and
c) optionally, a polymer over-coating on said film coating.

15. The oral pharmaceutical composition of claim 14 wherein said single core comprises a proton pump inhibitor selected from the group consisting of lansoprazole, pantoprazole, rabeprazole, omeprazole, esomeprazole, leminoprazole or mixtures.

16. The oral composition of claim 14 in the form of tablet containing lansoprazole.

17. The oral composition of claim 14 in the form of tablet containing pantoprazole.

18. The oral pharmaceutical composition of claim 14 wherein said enteric coating comprises a polymer selected from cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers and methacrylic acid polymers and copolymers or combinations thereof.

19. A process for preparing an oral pharmaceutical composition in a solid dosage form comprising:
a) forming single core comprising a proton pump inhibitor and a lubricant, wherein said single core has an exterior surface;
b) applying a coating of an enteric coating comprising a polymer and a lubricant onto the exterior surface of said single core containing said proton pump inhibitor, and without forming a separating layer between said single core and said enteric coating; and
c) optionally, applying a polymer overcoating on said film coating.
